(19) United States
(12) Patent Application Publication
(10) Pub. No.: US 2018/0161204 A1
(43) Pub. Date: Jun. 14, 2018

(54) SURGICAL INSTRUMENT FOR ATTACHING SCLERAL BUCKLE IN RETINAL DETACHMENT TREATMENT

(71) Applicant: Hernan Gras, Houston, TX (US)

(72) Inventor: Hernan Gras, Houston, TX (US)

(21) Appl. No.: 15/835,092

(22) Filed: Dec. 7, 2017

Related U.S. Application Data
(60) Provisional application No. 62/431,940, filed on Dec. 9, 2016.

(51) Int. Cl. A61F 9/007; A61F 9/00

(52) U.S. Cl. CPC A61F 9/00727; A61F 2009/00865; A61F 9/00736; A61F 9/0017

(57) ABSTRACT

A surgical instrument and a method of repairing a retinal detachment in an eye are provided. The surgical instrument includes a free end and an operable end opposite to the free end for insertion around the eye through an entry aperture. The tip of the operable end includes a hook portion that is rotated around a circumference of the eye upon insertion. Accordingly, the instrument allows for decreased procedure time and also decreased inflammation and trauma to the eye.

### RELATED APPLICATIONS

The present application claims the benefit of U.S. Provisional Application No. 62/431,940 filed Dec. 9, 2016, which is incorporated herein by reference in its entirety.

### BACKGROUND

### 1. Technical Field

The present disclosure relates to a surgical instrument and method of repairing a retinal detachment, and more particularly, to a surgical instrument with a hook portion used to attach a scleral buckle for retinal detachment treatment, and a method thereof.

### 2. Description of the Related Art

Retinal detachment occurs when the retina of the eye separates from the posterior wall of the eye, that is, separates from its underlying supporting tissue caused by vitreous filling the center of the eye (e.g., leading to a retinal tear). Such a detachment can potentially cause severe visual impairment. Treatment of a retinal detachment involves a surgical procedure of reconnecting the retina with those supporting tissues.

Developed treatment methods include scleral buckling in which the conjunctiva and Tenon capsule are is separated from the sclera, the retinal tear is localized and sub retinal fluid is drained by cutting through the sclera and choroid (or in combination with a vitrectomy procedure discussed further herein below). That is, a plurality of incisions or holes are formed in the sclera to provide access for surgical tools to the retina and other tissues. A silicone material or similar material is then sutured over the sclera to push the eye wall inward and support the tear. In particular, after dissecting the four rectus muscles in the eye, as shown in FIG. 1 of the related art, a belt-shaped band is introduced around a patient's eye and specifically below the muscles. The band is then sutured in all four quadrants of the eye. FIG. 2 shows a closure of the entry apertures or incisions during the procedure according to the related art. Such a procedure, however, requires lengthy recovery time and causes substantial inflammation and trauma to the eye.

Additionally, the scleral buckling procedure is often combined with a vitrectomy procedure. During such a procedure, multiple instruments are placed inside the eye to infuse fluid, cut and remove tissue. This method increases the chances of curing the disease and treating the retinal detachment by manipulating the retina and allowing internal tamponade, air, gas or silicone oil to be infused into the eye. However, the vitrectomy procedure still requires a scleral buckle attachment, adding an additional 20 to 30 minutes of procedure time. In addition, as discussed above, such a procedure causes substantial inflammation and trauma to the eye.

### SUMMARY

The present disclosure provides a surgical instrument for retinal detachment repair in an eye which reduces inflammation and trauma caused to a patient's eye during a repair procedure and reduce the time required to perform the procedure.

According to one aspect of the present disclosure, a surgical instrument for retinal detachment in the eye includes a free end and an operable end that is opposite to the free end. The operable end is inserted around the eye and through an entry aperture. Additionally, a tip of the operable end includes a hook portion that is rotated around a circumference of the eye upon insertion. Specifically, the hook portion is inserted between the rectus muscles, conjunctiva, Tenon capsule and the sclera within the eye when rotated after insertion. A band is then inserted through the entry aperture and passes under the rectus muscles, conjunctiva, and Tenon capsule by the rotation of the hook portion in an opposite direction. A scleral buckle is attached to ends of the band at the entry aperture to lock the band in place under the rectus muscles, conjunctiva, and Tenon capsule.

According to another aspect of the present disclosure, the entry aperture may be created by a single incision in one quadrant of the eye. The hook portion of the instrument may have a diameter of about 21-25 mm and may be angled about 38° to 48° from a shaft portion of the instrument. In addition, the band may be made of silicone and may be attached to an end of the hook portion. The instrument itself may be made of various materials such as metal, plastic, silicone, wood, glass graphite, Kevlar, polyvinyl chloride (PVC), and Teflon.

In an exemplary embodiment of the present disclosure, the instrument may be manually or robotically inserted into the eye through the entry aperture. The insertion opens the conjunctiva of the eye about 1 to 7 mm and the hook portion is rotated around the circumference of the eye and is extracted through the entry aperture. The surgical instrument may further include a light source attached to the tip of the operable end thereof to illuminate a path of the hook portion during rotation. Additionally, an imaging device may be attached at the tip of the operable end to capture images of a path of the hook portion during rotation.

According to yet another aspect of the present disclosure, a method of repairing a retinal detachment in an eye is provided. The method may include forming an incision in the eye to create an entry aperture and inserting a hook portion of a surgical instrument around the eye and under a conjunctiva and Tenon capsule of the eye. The hook portion is attached at a tip of an operable end of the surgical instrument. Additionally, the method may include inserting the hook portion into the entry aperture. The hook portion may then be rotated around a circumference of the eye and between rectus muscles, conjunctiva, and Tenon capsule and a sclera within the eye. A band, attached to an end of the hook portion, may be inserted through the entry aperture and around the circumference of the eye under the rectus muscles, conjunctiva, and Tenon capsule by the rotation of the hooking portion in an opposite direction. A scleral buckle may be attached to the ends of the band to lock the band in place.

In an exemplary embodiment of the present disclosure, the method may further include suturing the incision upon extraction of the hook portion and upon locking of the band to close the entry aperture. In addition, the band may be specifically attached to the end of the hook portion by a single suture stitch.

Notably, the present invention is not limited to the combination of the suturing apparatus element as listed above and may be assembled in any combination of the elements as described herein.

Other aspects of the disclosure are disclosed infra.

### BRIEF DESCRIPTION OF THE DRAWINGS

The embodiments herein may be better understood by referring to the following description in conjunction with the accompanying drawings in which like reference numerals indicate identically or functionally similar elements, of which:

FIG. 1 illustrates a method of repairing a retinal detachment according to the related art;

FIG. 2 illustrates closure of entry apertures in a retinal detachment repair according to the related art;

FIGS. 3A-3B illustrate a view of a surgical instrument for retinal detachment treatment according to an exemplary embodiment of the present disclosure;

FIG. 4 illustrates a view of the hook portion of the surgical instrument rotated around the circumference of an eye according to an exemplary embodiment of the present disclosure;

FIG. 5 illustrates a detailed view of the surgical instrument according to an exemplary embodiment of the present disclosure;

FIGS. 6A-6B illustrate a detailed view of the hook portion of the surgical instrument according to an exemplary embodiment of the present disclosure;

FIG. 7 illustrates a detailed view of the bottom of the surgical instrument according to an exemplary embodiment of the present disclosure;

FIG. 8 illustrates a method of repairing a retinal detachment in an eye according to an exemplary embodiment of the present disclose; and

FIG. 9 illustrates a flowchart of method of repairing a retinal detachment in an eye according to an exemplary embodiment of the present disclosure.

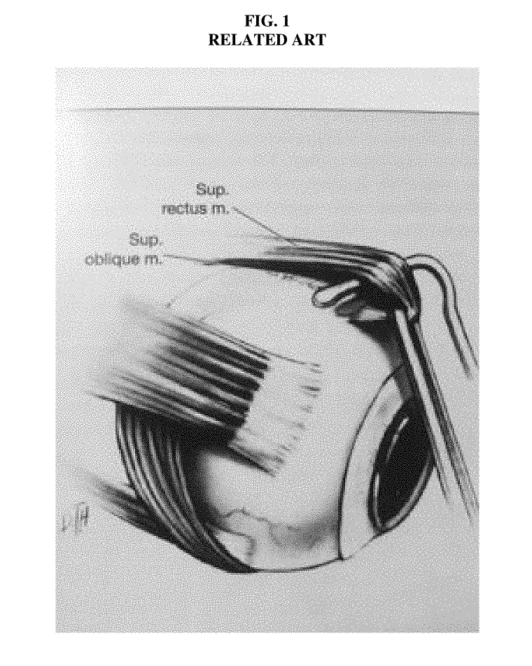

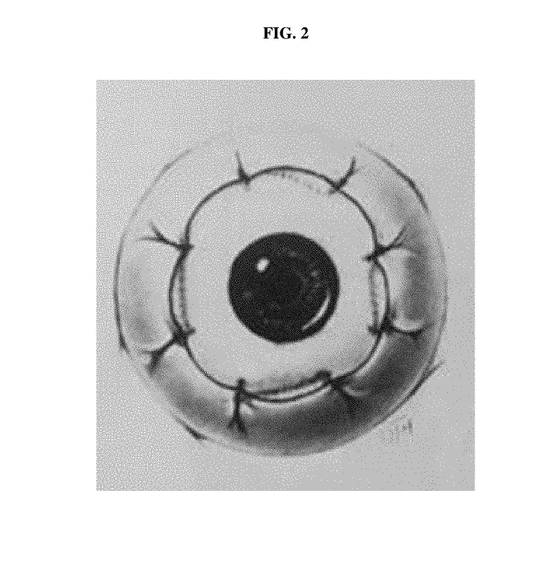

It should be understood that the appended drawings are not necessarily to scale, presenting a somewhat simplified representation of various features illustrative of the basic principles of the disclosure. The specific design features of the present disclosure as disclosed herein, including, for example, specific dimensions, orientations, locations, and shapes will be determined in part by the particular intended application and use environment.

### DETAILED DESCRIPTION

The presently disclosed subject matter will be described more fully hereinafter with reference to the accompanying drawings, in which some, but not all embodiments of the inventions are shown. The presently disclosed subject matter may be embodied in many different forms and should not be construed as limited to the embodiments set forth herein; rather these exemplary embodiments are provided so that this disclosure will satisfy applicable legal requirements. Indeed, many modifications and other exemplary embodiments of the presently disclosed subject matter set forth herein will come to mind to one skilled in the art to which the presently disclosed subject matter pertains, having the benefit of the teachings presented in the foregoing descriptions and the associated drawings. Therefore, it is to be understood that the presently disclosed subject matter is not limited to the specific embodiments disclosed and that modifications and other exemplary embodiments are intended to be included within the scope of the appended claims.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

The present disclosure provides a surgical instrument for retinal detachment repair in an eye that reduces the amount of suturing required for a surgical procedure by requiring only a single incision into the conjunctiva and Tenon capsule (e.g., fascia bulbi) of a sclera for implantation of a scleral band and buckle. That is, a hook portion of the instrument introduces a scleral band through an incision in the conjunctiva and under rectus muscles, conjunctiva, and Tenon capsule without opening the entire conjunctiva or dissecting the rectus muscles. The single incision and simplified implantation of the band using a hook portion of the instrument substantially reduces the inflammation and trauma caused to the eye during the surgical procedure. Additionally, the time required to perform the procedure is substantially reduced.

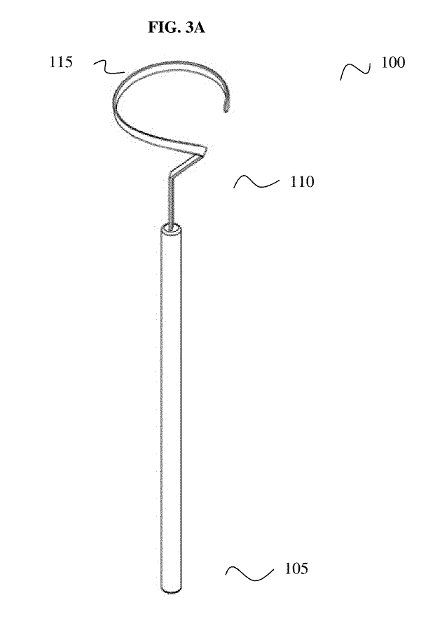

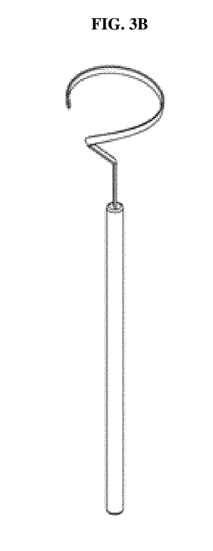

As shown in FIG. 3A, in one aspect, the present disclosure provides a surgical instrument 100 for retinal detachment repair in an eye that may include a free end 105 and an operable end 110 that is opposite to the free end 105. In particular, a portion of the operable end 110 may be inserted around the eye through an entry aperture, to be described in further detail hereinbelow. The entry aperture may be specifically formed by an incision in the conjunctiva and the Tenon capsule. A tip of the operable end 110 includes a hook portion 115 which may be rotated around a circumference of the eye upon insertion into the entry aperture. The hook portion 115 may be rotated either clockwise or counterclockwise. The hook portion, capable of being rotating around the eye's circumference, eliminates the need to make multiple incisions for multiple entry apertures. The entry aperture may thus be created by a single incision in any one of the four quadrants of the eye and specifically in the conjunctiva and the Tenon capsule. Notably, the hook portion may be formed as either a left tool as shown in FIG. 3A or as a right tool as shown in FIG. 3B.

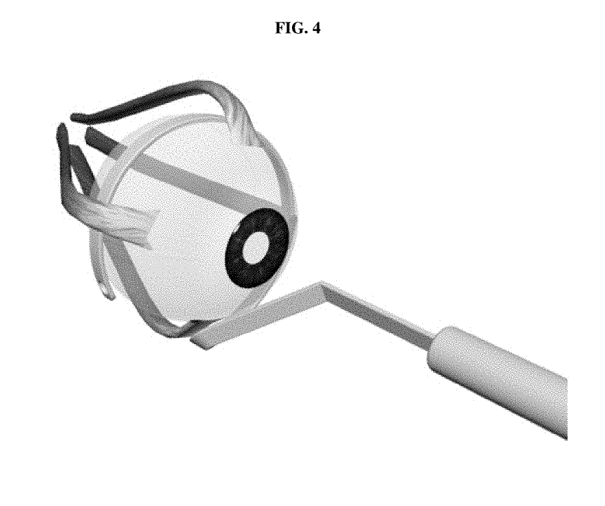

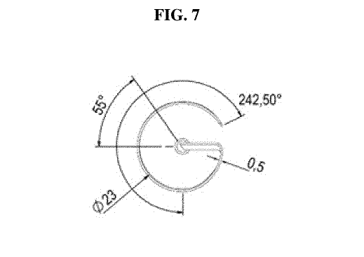

Further, as shown in FIG. 4, the hook portion may specifically be inserted between the rectus muscles, conjunctiva, and Tenon capsule, and the sclera within the eye when rotated after insertion. The insertion of the hook portion into the entry aperture opens the conjunctiva of the eye about 1 to 7 mm. In addition, a band may be attached to the hook portion such that when the hook portion rotates around the eye, the band is introduced therewith. For example, the band may be attached to the hook portion using a single suture stitch. That is, the tip of the hook portion may include an aperture (see 605 in FIG. 7B) through which the band may be attached. Alternately, the band may be attached to the hook portion by a button like connection. For example, an end of the band may include a protrusion fitted into the aperture of the hook portion.

Particularly, the band is inserted through the entry aperture and passes under the rectus muscles, conjunctiva, and Tenon capsule based on the rotation of the hook portion in an opposite direction. The band may be made of silicone, but is not limited thereto. Once the end of the band (e.g., the end passed around the eye) reaches back to the entry aperture, a scleral buckle may be attached to the both ends of the band to lock the band in place. Various known techniques may be used to lock the band in place using the buckle, for example, the buckle may be sutured or hooked.

Moreover, the scleral buckle may be a piece of silicone, rubber, or other similar material which then pushes the sclera in toward the middle of the eye. A vitrectomy procedure may then be executed once the buckle is in place to thus form a seal between the retina and the supporting tissue from which it has detached. The surgical instrument as described above with a hook portion thus allows for use of a small caliber in the vitrectomy procedure without sutures.

According to one embodiment, the instrument may either be manually inserted around the circumference of the eye or may be robotically inserted through the eye. For example, the instrument may be advanced toward the patient's eye by robotically steering the instrument. In such a scenario, the hook portion of the instrument may be replaced by a probe. Additionally, an imaging device may be included to provide a user with assistance in positioning and operating of the instrument (e.g., for echography, optical coherence tomography (OCT), radioscopy or the like). Accordingly, a surgeon would be capable of retaining high control using a minimally sized system to facilitate treatment of tissues within a patient. To further facilitate the process of inserting the hook portion and band around the eye, a light source may be disposed either along the length of the instrument (e.g., along a shaft of the instrument) or at the tip of the operable end of the instrument. The light source may illuminate a path of the hook portion during rotation to provide an improved viewing path for a surgeon.

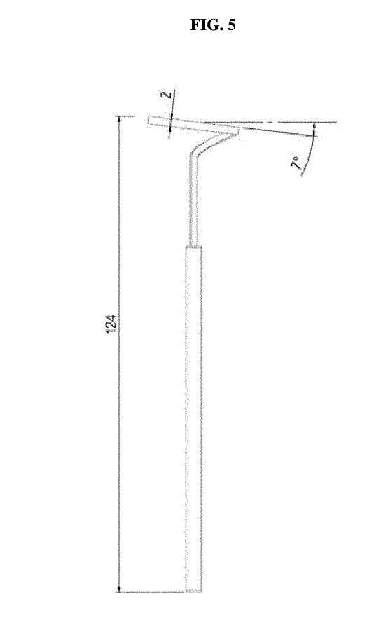

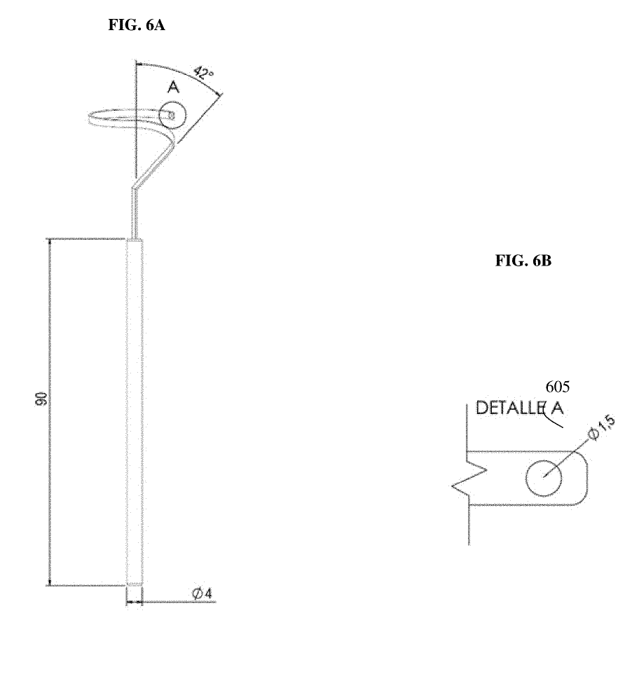

As shown in FIGS. 5-7, according to another embodiment, the instrument may have a total length of about 124 mm with a shaft portion thereof having a length of about 90 mm and a diameter of about 4 mm. The instrument may be made of various materials typically used for surgical instruments. For example, the instrument may be made of metal, plastic, silicone, wood, glass graphite, Kevlar, Teflon, PVC and the like, but is not limited thereto. The hook portion may have a cylindrical shape with a diameter ranging from about 21-25 mm, and preferably, 23 mm with a thickness of about 0.5 mm. The circular portion of the hook portion of the instrument may be offset from a horizontal axis by about 7° (e.g., the hook portion curvature) and from a vertical axis (e.g., with respect to the shaft) by about 38° to 48° , and preferably about 42° (e.g., the hook portion curvature). The particular angles may, however, vary based on the length of the overall instrument. Based on the bottom view of the hook portion shown in FIG. 7, the diameter of the circular shape formed by the hook portion may be about 23 mm. In addition, the aperture on the hook portion through which the band may be inserted to be attached to the hook portion may have a diameter of about 1.5 mm.

Notably, the surgical instrument of the present disclosure is not limited to the above described measurements. The measurements are merely exemplary to provide those of ordinary skill in the art with an understanding of the embodiment. However, the measurements may be adjusted by design choice and for varied applications.

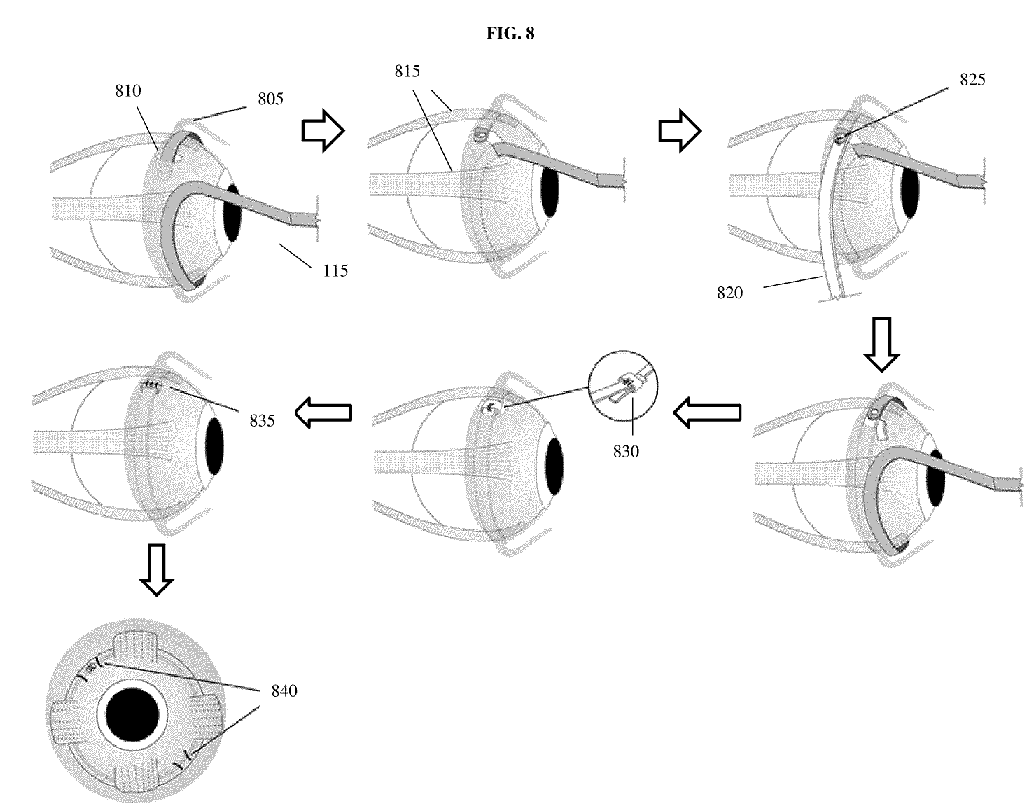

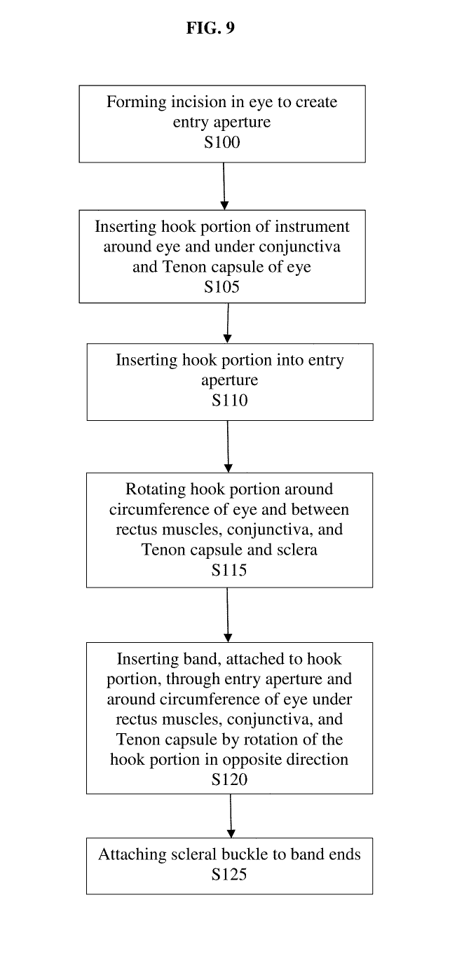

According to another aspect and as shown in FIG. 8, a method of repairing a retinal detachment of an eye using the surgical instrument described herein is provided. First, the hook portion 115 of the surgical instrument 100 may be inserted around the eye, under the conjunctiva 805 and Tenon capsule. The end of the hook portion may then be inserted into an entry aperture 810. Upon insertion, the hook portion 115 may be rotated around the circumference of the eye, under the rectus muscles 815 such that the hook portion is under the conjunctiva, Tenon capsule, and rectus muscles based on the rotation. Once the hook portion has rotated completely around the circumference of the eye and the end thereof reaches the entry aperture, a band 820 may be attached to the end of the hook portion 115. In particular, the band may be attached at the aperture 605 of the hook portion by a stitch 825, but is not limited thereto, and other attaching techniques may be used as described herein above. That is, the band 820 may be attached to the aperture 605 in any manner that holds the two components together. For example, the end of the band may include a protrusion that is press fitted into the aperture (e.g., a button like connection).

Additionally, once the band 820 is attached to the hook portion 115, the hook portion 115 may be rotated in an opposite direction to thus draw the band around the circumference of the eye and under the conjunctiva, Tenon capsule, and rectus muscles. This rotation of the hook portion in the opposite direction also extracts the hook portion from the entry aperture. In other words, the hook portion is rotated in the opposite direction until extracted at which point the end of the band also reaches the entry aperture. Once the hook portion is extracted, both ends of the band are accessible at the entry aperture and a scleral buckle 830 may be attached to the ends. The buckle 830 may then be sutured (or other closing technique) to lock the band in place (see 835). Further, the incision of the entry aperture may be sutured (or other closing technique) to complete the procedure (see 840 for incision closure).

Furthermore, FIG. 10 illustrates a flowchart of such a method. In particular, the method may include forming an incision in the eye to create an entry aperture (S100) and inserting a hook portion of a surgical instrument around the eye and under the conjunctiva and Tenon capsule of the eye (S105). The hook portion is then inserted into the entry aperture (S110). The incision in the present disclosure may be made in any of the four quadrants of the eye based on where a tear may be within the eye. In addition, the hook portion is attached to a tip of an operable end of the surgical instrument. The method may further include rotating the hook portion of the instrument around a circumference of the eye between rectus muscles, conjunctiva, and Tenon capsule and a sclera within the eye (S115). A band, attached to an end of the hook portion, may then be inserted through the entry aperture and around the circumference of the eye under the rectus muscles, the conjunctiva, and the Tenon capsule by the rotation of the hook portion in an opposite direction (S120). In other words, the band may be moved around the circumference of the eye by the rotation of the hook portion in the opposite direction. Lastly, the method may include attaching a scleral buckle to ends of the band to lock the band in place (S125).

According to one embodiment, the present disclosure may further include rotating the hook portion in the opposite direction to extract the hook portion through the entry aperture. In other words, once the hook portion reaches the entry aperture again after rotating around the circumference of the eye, the band may be released from the hook portion. The band may be attached to the hook portion by a single suture stitch or by alternate attaching techniques as discussed herein above. Since both ends of the band are at the entry aperture, the buckle may be attached to the ends of the band and then the incision or entry aperture may be sutured upon extraction of the hook portion to close the entry aperture. Accordingly, the process merely requires one small incision to be closed, thus causing less trauma and inflammation compared to the related art in which incisions are made in at least four quadrants of the eye.

As discussed above, the surgical instrument according to the present disclosure is capable of reducing the amount of time required to complete a retinal detachment repair procedure. Specifically, the introduction of a hook and band around the circumference of the eye may be reduced to about 2 to 4 minutes, a process which conventionally takes about 20 to 30 minutes. Additionally, by merely requiring a single incision to fasten a band around the eye, less wounds are required to be closed, thus reducing the trauma and inflammation to the eye.

The many features and advantages of the disclosure are apparent from the detailed specification, and thus, it is intended by the appended claims to cover all such features and advantages of the invention which fall within the true spirit and scope of the disclosure. Further, since numerous modifications and variations will readily occur to those skilled in the art, it is not desired to limit the invention to the exact construction and operation illustrated and described, and accordingly, al suitable modifications and equivalents may be resorted to, falling within the scope of the disclosure.

What is claimed is:

1. A surgical instrument for retinal detachment repair in an eye, comprising:
a free end; and
an operable end opposite to the free end for insertion around the eye through an entry aperture,
wherein a tip of the operable end includes a hook portion which is rotated around a circumference of the eye upon insertion.

2. The surgical instrument of claim 1, wherein the hook portion is inserted between rectus muscles, conjunctiva, Tenon capsule and a sclera within the eye when rotated after insertion.

3. The surgical instrument of claim 2, wherein a band is inserted through the entry aperture and passes under the rectus muscles, conjunctiva, and Tenon capsule by the rotation of the hook portion in an opposite direction.

4. The surgical instrument of claim 3, wherein a scleral buckle is attached to ends of the band at the entry aperture to lock the band in place under the rectus muscles, conjunctiva, and Tenon capsule.

5. The surgical instrument of claim 1, wherein the entry aperture is created by a single incision in one quadrant of the eye.

6. The surgical instrument of claim 1, wherein the hook portion has a diameter of about 21 mm to 25 mm and is angled about 38° to 48° from a shaft portion of the instrument.

7. The surgical instrument of claim 3, wherein the band is made of silicone and is attached to an end of the hook portion.

8. The surgical instrument of claim 1, wherein the instrument is made of metal, plastic, silicone, wood, glass graphite, Kevlar, Teflon, or polyvinyl chloride.

9. The surgical instrument of claim 1, wherein the instrument is manually or robotically inserted into the eye through the entry aperture.

10. The surgical instrument of claim 1, wherein the insertion opens the conjunctiva of the eye about 1 to 7 mm and the hook portion is rotated around the circumference of the eye and extracted through the entry aperture.

11. The surgical instrument of claim 1, further comprising:
a light source attached at the tip of the operable end to illuminate a path of the hook portion during rotation.

12. A method of repairing a retinal detachment in an eye, comprising:
forming an incision in the eye to create an entry aperture;
inserting a hook portion of a surgical instrument around the eye and under a conjunctiva and Tenon capsule of the eye, wherein the hook portion is attached at a tip of an operable end of the surgical instrument;
inserting the hook portion into the entry aperture;
rotating the hook portion of the surgical instrument around a circumference of the eye and between rectus muscles, the conjunctiva, the Tenon capsule, and a sclera within the eye;
inserting a band, attached to an end of the hook portion, at the entry aperture and around the circumference of the eye under the rectus muscles, the conjunctiva, and Tenon capsule by the rotation of the hook portion in an opposite direction; and
attaching a scleral buckle to ends of the band to lock the band in place.

13. The method of claim 12, further comprising:
suturing the incision upon extraction of the hook portion and upon locking of the band to close the entry aperture.

14. The method of claim 12, wherein the hook portion has a diameter of about 21 mm to 25 mm and is angled about 38° to 48° from a shaft portion of the instrument.

15. The method of claim 12, wherein the band is made of silicone and is attached to the hook portion by a single suture stitch.

16. The method of claim 12, wherein the instrument is manually or robotically inserted into the eye through the entry aperture.

17. The method of claim 12, wherein the insertion opens the conjunctiva of the eye about 1 to 7 mm.

18. The method of claim 12, wherein a light source is attached at the tip of the operable end to illuminate a path of the hook portion during rotation.

19. The method of claim 11, wherein an imaging device is attached at the tip of the operable end to capture images of a path of the hook portion during rotation.
